(19) United States
(12) Patent Application Publication
(10) Pub. No.: US 2017/0304176 A1
(43) Pub. Date: Oct. 26, 2017

(54) ANTI-COLOR FADING AND FRAGRANCE PROTECTION COMPOSITIONS

(71) Applicant: 3 V INCORPORATED, Georgetown, SC (US)

(72) Inventors: Giorgio ZANCHI, Daniel Island, SC (US); Paul Maria SECCOMANDI, Georgetown, SC (US)

(21) Appl. No.: 15/520,202

(22) PCT Filed: Oct. 21, 2015
(86) PCT No.: PCT/US2015/056612

§ 371 Date: Apr. 19, 2017

Related U.S. Application Data
(60) Provisional application No. 62/066,444, filed on Oct. 21, 2014.

(51) Int. Cl. A61K 8/49; A61Q 17/04; A61K 8/35; A61K 8/37

(52) U.S. Cl. CPC A61K 8/4966; A61K 8/37; A61Q 17/04; A61K 8/35; A61K 2800/5922; A61K 2800/52

(57) ABSTRACT

The present disclosure is directed to stabilizing compositions comprising a mixture of three or more ultraviolet filters. The disclosed compositions are useful for stabilizing the color and the fragrance of formulations, including cosmetic formulations and household products.

### TECHNOLOGICAL FIELD

The present disclosure relates to compositions for stabilizing the color and the fragrance of formulations, including cosmetic formulations and household products.

### BACKGROUND

Particularly in the field of cosmetics and household products, many formulations include dyes (natural or synthetic), fragrances (natural or synthetic) which are generally intended to improve the appearance, to impart a characteristic color and/or to impart a characteristic odor. Staining can also result due to the intrinsic color of some ingredients, for example perfumes and fragrances. The color of the formulation is due to the presence of the compounds that selectively absorb light.

Often such colored and perfumed formulations are sold in packaging transparent to light, such as glass or plastic, so the content is exposed to light. Such formulations are often sold as cosmetic formulation or household product formulations or cosmetic or house product precursor formulations. Such packaging is generally also transparent to ultraviolet (UV). Exposure to ultraviolet light (between 200 nm and 400 25 nm) may induce a change that modifies the chemical structure of the dyes and the fragrances. Consequently, the color of the formulation can change or transfer. In addition, the intensity and the tone of the fragrance can change or transfer as well. In particular, the following conditions may be observed: a change of the perfume, a discoloration (e.g. a formulation yellowing, turning pink, etc.), flocculation and precipitation of substances, or clouding. Such changes in condition may adversely affect the customer's perception of the product comprising the formation. To improve the stability of the colored and perfumed formulations, UV filters may be used which provide protection by selective absorption of UV radiation.

Ultraviolet absorbing compounds are known, such as those mentioned for example in Annex VII of the EEC Directive No. 76/168 (relating to cosmetic UV filters accepted) or in the Guidelines of the FDA (American Food and Drugs Administration), namely in the Federal Register, Vol. 43, No. 166 (Aug. 25, 1978). These compounds, however, are intended to protect the skin and hair against the harmful effects of UV radiation, and not the cosmetics themselves or their components such as dyes and fragrances.

It is known that the use of two UV filters will not protect a perfume or a dye from degradation by UV rays. Thus, ternary or quaternary mixtures of UV filters have been proposed in order to guarantee the stabilization of perfumes or dyes. For example, it was shown in application FR 2 916 346 that the color and the odor of a toilet water comprising 1% wt of a mixture of n-hexyl 2-(4-diethylamino-2-hydroxybenzoyl)benzoate and ethylhexyl methoxycinnamate 35/65 are degraded on exposure to UV and heat, so that it is necessary to add a third UV filter of the butylmethoxy-dibenzoylmethane type to obtain effective protection of the product against UV.

Moreover, the color and the odor of a toilet water comprising 1% wt of ethylhexylmethoxycinnamate and 0.3% wt of butylmethoxydibenzoylmethane are degraded upon exposure to UV and heat. WO2006/005846 discloses adding a third filter as a salicylate derivative. Ethylhexylsalicylate has been added to ethylhexylmethoxycinnamate and to butylmethoxydibenzoylmethane to protect cosmetic compositions from discoloration when they are exposed to UV.

FR 2 916 347 and FR 2 923 385 also describe quaternary mixtures of UV filters to stabilize the color and the odor of cosmetics compositions. These quaternary mixtures comprise a cinnamate, a dibenzoylmethane and a salicylate derivative, in combination with either a derivative of hydroxyaminobenzophenone, or a derivative of benzotriazole.

However, ethylhexylsalicylate has the drawback of being odorous. It releases a greasy, pyrogenous, plastic, rubber note, so that its use for the stabilization of fragrances and dyes it is not satisfactory.

Furthermore, it has been proposed to add, to a mixture of UV filters comprising a cinnamate and a dibenzoylmethane, an organic compound and nitroxyl or hydroxylamine group such as tris-tetramethylhydroxypiperidinol (marketed as TINOGARD Q) for protecting fabrics, cosmetic compositions and household maintenance products from the effects of light, heat and oxidation (WO 2005/042828).

Finally, U.S. Patent Publication No. US 2103/0045913 discloses the addition of non-volatile oils to UV filter mixtures of a derivative of cinnamate and a derivative of dibenzoylmethane. However, these oils do not photostibilize the disclosed UV filter mixture.

Unfortunately the stabilizing additives recommended in the art cited above contain large amounts of UV filters, which are raw materials expensive and tend to alter the color or the scent of the cosmetic compositions.

Increasing the quantity of UV filters in the cosmetic compositions has also been tried, in order to protect the products against UV radiation; however this route was not successful, because at higher concentration the film of the cosmetic composition leaves on the skin greasy sensation and because regulatory requirements impose maximum limits to the use of each UV filter.

Therefore, a solution to the stabilization of cosmetic compositions and household products that do not have the drawbacks of the prior art, including stabilizing mixtures that do not alter the organoleptic properties such as odor and color of the cosmetic compositions, while providing sufficient stabilization to the product exposed to daylight or to temperature differences would be highly advantageous.

### SUMMARY OF THE INVENTION

Aspects of the present disclosure are directed to compositions and methods for making compositions that ensure the stabilization of staining (e.g. color) and perfume (e.g. fragrance) in cosmetic formulations, while using significantly less UV absorber mixtures compared to known water-based and non-water-based UV stabilizers. Such compositions would be useful as components in an end use composition having desired color or fragrances such as, for example, a cosmetic formulation, household product formulations or cosmetic precursor formulation, house product precursor formulation, etc.

Aspects of the present disclosure contemplate compositions and methods for making compositions to ensure a significantly longer protection against the UVA, UVB and UVC radiations compared to known compositions incorporated into known non-water-based and water-based formulations.

In addition, aspects of the present disclosure provide compositions with higher photostability compared to known-water-based and water-based formulations.

According to one aspect a method for stabilizing the color and fragrance of a cosmetic against the effect of UV radiation is disclosed comprising the steps of preparing a UV absorbing composition comprising:
(a) at least one cinnamate-containing compound or cinnamate-containing derivative compound, said cinnamate-containing compound or cinnamate-containing derivative compound absorbing radiation in the UV region;
(b) at least one triazine-containing compound or triazine-containing derivative compound, said triazine-containing compound or triazine-containing derivative compound absorbing radiation in the UVB region; and
(c) at least one dibenzoylmethane-containing compound or dibenzoylmethane-containing derivative compound, said dibenzoylmethane-containing compound or dibenzoylmethane-containing derivative compound absorbing radiation in the UV region; and
   incorporating the UV absorbing composition into a cosmetic formulation.

In a further aspect, the UV absorbing composition comprises about 5.0 to about 90.0% by weight of compound (a); about 5.0 to about 90.0% by weight of compound (b); and about 5.0 to about 90.0% by weight of compound (c).

In another aspect, compound (a) comprises Uvasorb HEB, CAS [154702-15-5]; compound (b) comprises Octyl methoxycinnamate, CAS [5466-77-3]; and compound (c) comprises Avobenzone, CAS [70356-09-1].

In another aspect, the UV absorbing composition further comprises (d) one or more surfactants.

In yet another aspect, the UV absorbing composition comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

In still another aspect, the composition comprises a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Avobenzone, CAS [70356-09-1]; and about 20.0 to about 80.0% by weight of compound (d).

A further aspect, is directed to a method for stabilizing the color and fragrance of a cosmetic against the effect of UV radiation comprising the steps of preparing a UV absorbing composition comprising:
(a) at least one UV absorbing compound, said UV absorbing compound comprising a cinnamate-containing compound or a cinnamate-containing derivative compound;
(b) at least one UV absorbing compound absorbing in the UVB region, said UV absorbing compound absorbing in the UVB region comprising a triazines-containing compound or a triazine-containing derivative compound; and
(c) at least one UV absorber compound absorbing in the UVA region, said UV absorber compound absorbing in the UVA region comprising a triazine-containing compound or a triazine derivative compound; and
   incorporating the UV absorbing composition into a cosmetic formulation.

In a further aspect, the UV absorbing composition comprises a combination of: about 5.0 to about 90.0% by weight of compound (a); about 5.0 to about 90.0% by weight of compound (b); and about 5.0 to about 90.0% by weight of compound (c).

In yet another aspect, compound (a) comprises Uvasorb HEB, CAS [154702-15-5]; compound (b) comprises Octyl methoxycinnamate, CAS [5466-77-3]; and compound (c) comprises Uvasorb K2A, CAS [288254-16-0].

In a still further aspect, the UV absorbing composition further comprises: (d) at least one surfactant.

In yet another aspect, the UV absorbing compound comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

In a still further aspect, the composition comprises a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or a mixture of surfactants.

A further aspect of the present disclosure is directed to a composition, comprising:
(a) at least one UV absorbing compound comprising a cinnamate-containing compound or a cinnamate-containing derivative compound;
(b) at least one UV absorbing compound absorbing in the UVB region, said UV absorbing compound absorbing in the UVB region comprising a triazine-containing compound or a triazine-containing derivative compound; and
(c) at least one UV absorbing compound comprising a dibenzoylmethane-containing compound or a dibenzoylmethane-containing derivative compound.

In another aspect, the composition comprises a combination of: about 5.0 to about 90.0% by weight of compound (a); about 5.0 to about 90.0% by weight of compound (b); and about 5.0 to about 90.0% by weight of compound (c).

In yet another aspect, compound (a) comprises Uvasorb HEB, CAS [154702-15-5]; compound (b) comprises Octyl methoxycinnamate, CAS [5466-77-3]; and compound (c) comprises Avobenzone, CAS [70356-09-1].

In a further aspect, the composition further comprises:
(d) one or more surfactants.

A still further aspect of the present disclosure is directed to a composition, comprising:
(a) at least one UV absorbing compound comprising a cinnamate-containing compound or cinnamate-containing derivative compound,
(b) at least one UV absorbing compound absorbing radiation in the UVB region, said UV absorbing compound absorbing radiation in the UVB region comprising a triazine-containing compound or a triazine-containing derivative compound; and
(c) at least one UV absorbing compound absorbing radiation in the UVA region, said UV absorbing compound absorbing radiation in the UVA region comprising a triazine-containing compound or a triazine-containing derivative compound.

In a further aspect, the composition further comprises (d) one or more surfactants.

In yet another aspect, the composition comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

In a another aspect, a composition comprises a combination of about 5.0 to about 90.0% by weight of compound (a); about 5.0 to about 90.0% by weight of compound (b); and about 5.0 to about 90.0% by weight of compound (c).

In yet another aspect, compound (a) comprises Uvasorb HEB, CAS [154702-15-5]; compound (b) comprises Octyl methoxycinnamate, CAS [5466-77-3]; and compound (c) compound Uvasorb K2A, CAS [288254-16-0].

A further aspect of the present disclosure is directed to a composition, comprising:
(a) at least one UV absorbing compound comprising a cinnamate-containing compound or a cinnamate-containing derivative compound;
(b) at least one UV absorbing compound absorbing UV radiation in the UVB region, said UV absorbing compound absorbing UV radiation in the UVB region comprising a triazine-containing compound or a triazine-containing derivative compound; and
(c) at least one UV absorbing compound comprising a dibenzoylmethane-containing compound or a dibenzoylmethane-containing derivative compound.

In another aspect, the composition further comprises (d) one or more surfactants.

In a still further aspect, the composition comprises: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Avobenzone, CAS [70356-09-1]; and about 20.0 to about 80.0% by weight of compound (d).

Another aspect of the present disclosure is direct to a composition, comprising:
(a) at least one UV absorbing compound comprising a cinnamate-containing compound or a cinnamate-containing derivative compound;
(b) at least one UV absorbing compound absorbing radiation in the UVB region, said UV absorbing compound absorbing radiation in the UVB region comprising a triazine-containing compound or a triazine-containing derivative compound; and
(c) at least one UV absorbing compound absorbing radiation in the UVA region, said UV absorbing compound absorbing radiation in the UVA region comprising a triazine-containing compound or a triazine-containing derivative compound.

In a further aspect, the composition further comprises (d) one or more surfactants.

In yet another aspect, the composition comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

In yet another aspect, the composition comprises a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or more surfactants.

A still further aspect is directed to a cosmetic formulation or a cosmetic precursor formulation comprising a composition, comprising:
(a) at least one UV absorbing compound comprising a cinnamate-containing compound or a cinnamate-containing derivative compound;
(b) at least one UV absorbing compound absorbing radiation in the UVB region, said UV absorbing compound absorbing radiation in the UVB region comprising a triazine-containing compound or a triazine-containing derivative compound; and
(c) at least one UV absorbing compound absorbing radiation in the UVA region, said UV absorbing compound absorbing radiation in the UVA region comprising a triazine-containing compound or a triazine-containing derivative compound.

A further aspect is directed to a cosmetic formulation of cosmetic precursor formulation comprising a composition comprising about 1.0 to about 50.0% by weight of compound (a); about 1.0 to about 50.0% by weight of compound (b); about 1.0 to about 50.0% by weight of compound (c); and about 50.0 to about 90.0% by weight of one or more surfactants.

A further aspect is directed to a cosmetic formulation of cosmetic precursor formulation comprising a composition comprising a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or more surfactants.

A still further aspect is directed to a household product formulation or household product precursor formulation comprising a composition, comprising:
(a) at least one UV absorbing compound comprising a cinnamate-containing compound or a cinnamate-containing derivative compound;
(b) at least one UV absorbing compound absorbing radiation in the UVB region, said UV absorbing compound absorbing radiation in the UVB region comprising a triazine-containing compound or a triazine-containing derivative compound; and
(c) at least one UV absorbing compound absorbing radiation in the UVA region, said UV absorbing compound absorbing radiation in the UVA region comprising a triazine-containing compound or a triazine-containing derivative compound.

A still further aspect is directed to a household product formulation or household product precursor formulation comprising a composition, comprising a composition comprising about 1.0 to about 50.0% by weight of compound (a); about 1.0 to about 50.0% by weight of compound (b); about 1.0 to about 50.00% by weight of compound (c); and about 50.0 to about 90.0% by weight of one or more surfactants.

A still further aspect is directed to a household product formulation or household product precursor formulation comprising a composition, comprising a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or more surfactants.

### DETAILED DESCRIPTION

According to one aspect, the present disclosure provides a stabilizer composition comprising a combination of at least one UV filter derivative of s-triazine (defined as COMPONENT A) not known to have been used in combination with one or more UV filter compounds defined as COMPONENT B.

Preferred compounds for COMPONENT A comprise:

Uvasorb HEB, CAS [154702-15-5]

Preferred compounds for COMPONENT B comprise:

p-Aminobenzoic Acid, CAS [150-13-0]

Padimate O, CAS [21245-02-3]

Ensulizole, CAS [27503-81-7]

Cinoxate, CAS [104-28-9]

Dioxybenzone, CAS [131-53-3]

Oxybenzone, CAS [131-57-7]

Homosalate, CAS [118-56-9]

Menthyl anthranilate, CAS [134-09-8]

Octocrylene, CAS [6197-30-4]

Octyl methoxycinnamate, CAS [5466-77-3]

Octyl salicylate, CAS [118-6-5]

Sulisobenzone, CAS [4065-45-6]

Trolamine salicylate, CAS [2174-16-5]

Avobenzone, CAS [70356-09-1]

Ecamsule, CAS [92761-26-7]

Titanium dioxide, CAS [13463-67-7]

Zinc oxide, CAS [1314-13-2]

4-Methylbenzylidene camphor, CAS [36861-47-9]

Tinosorb M, CAS [103597-45-1]

Bemotrizinol, CAS [187393-00-6]

Bisdisulizole disodium, CAS [180898-37-7]

Mexoryl XL, CAS [155633-54-8]

Uvinul DS 49, CAS [3121-60-6]

Uvinul T150, CAS [88122-99-0]

Uvinul A Plus, CAS [302776-68-7]

Amiloxate, CAS [71617-10-2]

Uvasorb K2A, CAS [288254-16-0]

Parsol LSX,

Derivative of the PABA,

Derivative of the benzimidazole,

Derivative of the cinnamic acid,

Derivative of the benzophenone,

Derivative of the salicylic acid,

Derivative of the anthranilic acid,

Derivative of the cyanoacrylic acid,

Derivative of the dibenzoylmethane,

Derivative of the terephthalidene dicamphor,

Derivative of the benzylidene camphor,

Derivative of the benzotriazole,

Derivative of the s-triazine,

Derivative of the silicone and

Derivative of the acrylic acid.

Aspects of the present disclosure therefore provide stabilizer compositions as follows:

Composition 1

Aspects of the present disclosure contemplate a mixture of one COMPONENT A compound with one COMPONENT 1 compound comprising the following ranges:

COMPONENT A—5.0-90.0% of a s-triazine derivative and

COMPONENT B—5.0-90.0% of a s-triazine derivative.

In a further aspect. COMPOSITION 1 comprises:

COMPONENT A—5.0-90.0% of Uvasorb HEB, CAS [154702-15-5] and

COMPONENT B—5.0-90.0% of Uvasorb K2A, CAS [288254-16-0].

In another aspect, COMPOSITION 1 comprises:

COMPONENT A—70.0% of Uvasorb HEB, CAS [154702-15-5] and

COMPONENT B—30.0% of Uvasorb K2A, CAS [288254-16-0].

Composition 2

Aspects of the present disclosure contemplate a mixture of one COMPONENT A compound with two COMPONENT B compounds comprising the following ranges:

COMPONENT A—5.0-90.0% of a s-triazine derivative,

COMPONENT B—5.0-90.0% of a s-triazine derivative and

COMPONENT B—5.0-90.0% of a cinnamate derivative.

One further aspect contemplates COMPOSITION 2 comprising:

COMPONENT A—5.0-90.0% of Uvasorb HEB, CAS [154702-15-5],

COMPONENT B—5.0-90.0% of Uvasorb K2A, CAS [288254-16-0] and

COMPONENT B—5.0-90.0% of Octyl methoxycinnamate, CAS [5466-77-3].

In a still further aspect COMPOSITION 2 comprises:

COMPONENT A—15.0% of Uvasorb HEB, CAS [154702-15-5],

COMPONENT B—15.0% of Uvasorb K2A, CAS [288254-16-0] and

COMPONENT B—70.0% of Octyl methoxycinnamate, CAS [5466-77-3].

Composition 3

Aspects of the present disclosure contemplate a mixture of one COMPONENT A compound with two COMPONENT B compounds comprising the following ranges:

COMPONENT A—5.0-90.0% of a s-triazine derivative,

COMPONENT B—5.0-90.0% of a dibenzoylmethane derivative and

COMPONENT B—5.0-90.0% of a cinnamate derivative.

In one aspect, COMPOSITION 3 comprises:

COMPONENT A—5.0-90.0% of Uvasorb HEB, CAS [154702-15-5],

COMPONENT B—5.0-90.0% of Avobenzone, CAS [70356-09-1] and

COMPONENT B—5.0-90.0% of Octyl methoxycinnamate, CAS [5466-77-3].

In a further aspect, COMPOSITION 3 comprises:

COMPONENT A—20.0% of Uvasorb HEB, CAS [154702-15-5],

COMPONENT B—20.0% of Avobenzone, CAS [70356-09-1] and

COMPONENT B—60.0% of Octyl methoxycinnamate, CAS [5466-77-3].

Composition 4

Aspects of the present disclosure further contemplate a mixture of one COMPONENT A compound with three COMPONENT B compounds comprising the following ranges:

COMPONENT A—5.0-90.0% of a s-triazine derivative,

COMPONENT B—5.0-90.0% of a dibenzoylmethane derivative,

COMPONENT B—5.0-90.0% of a cinnamate derivative and

COMPONENT B—5.0-90.0% of a s-triazine derivative.

In a further aspect, COMPOSITION 4 comprises:

COMPONENT A—5.0-90.0% of Uvasorb HEB, CAS [154702-15-5],

COMPONENT B—5.0-90.0% of Avobenzone, CAS [70356-09-1],

COMPONENT B—5.0-90.0% of Octyl methoxycinnamate, CAS [5466-77-3],

COMPONENT B—5.0-90.0% of Uvasorb K2A, CAS [288254-16-0].

In another aspect, COMPOSITION 4 comprises:

COMPONENT A—20.0% of Uvasorb HEB, CAS [154702-15-5],

COMPONENT B—5.0% of Avobenzone, CAS [70356-09-1],

COMPONENT B—65.0% of Octyl methoxycinnamate, CAS [5466-77-3],

COMPONENT B—10.0% of Uvasorb K2A, CAS [288254-16-0].

Composition 5

Aspects of the present disclosure contemplate a mixture of one COMPONENT A with three COMPONENT B comprising the following ranges:

COMPONENT A—5.0-90.0% of a s-triazine derivative,

COMPONENT B—5.0-90.0% of a benzophenone derivative,

COMPONENT B—5.0-90.0% of a cinnamate derivative and

COMPONENT B—5.0-90.0% of a s-triazine derivative.

In a further aspect, COMPOSITION 5 comprises:

COMPONENT A—5.0-90.0% of Uvasorb HEB, CAS [154702-15-5],

COMPONENT B—5.0-90.0% of Uvinul A Plus, CAS [302776-68-7].

COMPONENT B—5.0-90.0% of Octyl methoxycinnamate, CAS [5466-77-3],

COMPONENT B—5.0-90.0% of Uvasorb K2A, CAS [288254-16-0].

In another aspect, COMPOSITION 5 comprises:

COMPONENT A—10.0% of Uvasorb HEB, CAS [154702-15-5],

COMPONENT B—10.0% of Uvinul A Plus, CAS [302776-68-7],

COMPONENT B—70.0% of Octyl methoxycinnamate, CAS [5466-77-3],

COMPONENT B—10.0% of Uvasorb K2A, CAS [288254-16-0].

The above compositions are prepared as follows:

### Example 1

We prepared a stabilizing composition comprising 70% of Uvasorb HEB by mass and 30% of Uvasorb K2A by mass, according to the following protocol. In a nitrogen blanketed flask, 70 g of Uvasorb HEB was loaded with heat is applied to reach 140° C. once the Uvasorb HEB was molten and fluid a gentle agitation was started. A 30 g sample of Uvasorb K2A was loaded. The mixture was heated and maintained at 140° C. with stirring until obtaining a clear liquid. The homogeneous mixture was flaked and milled obtaining a whitish powder.

### Example 2

A stabilizing composition was prepared comprising 70% of Octyl methoxy cinnamate by mass, 15% of Uvasorb HEB by mass and 15% of Uvasorb K2A by mass, according to the following protocol. In a flask, were introduced 70 g Octyl methoxy cinnamate, 15 g of Uvasorb HEB and 15 g of Uvasorb K2A. The mixture thus obtained was heated to 60° C. with stirring until obtaining a clear liquid.

### Example 3

A stabilizing composition was prepared comprising 60% of Octyl methoxy cinnamate by mass, 20% of Uvasorb HEB by mass and 20% of Avobenzone by mass, according to the following protocol. In a flask, were introduced 60 g Octyl methoxy cinnamate, 20 g of Uvasorb HEB and 20 g of Avobenzone. The mixture thus obtained was heated to 60° C. with stirring until obtaining a clear liquid.

### Example 4

A stabilizing composition was prepared comprising 65% of Octyl methoxy cinnamate by mass, 20% of Uvasorb HEB by mass, 10% of Uvasorb K2A and 5% of Avobenzone by mass, according to the following protocol. In a flask, were introduced 65 g Octyl methoxy cinnamate, 20 g of Uvasorb HEB, 10 g of Uvasorb K2A and 5 g of Avobenzone. The mixture thus obtained was heated to 60° C. with stirring until obtaining a clear liquid.

### Example 5

A stabilizing composition was prepared comprising 70% of Octyl methoxy cinnamate by mass, 10% of Uvasorb HEB by mass, 10% of Uvasorb K2A and 10% of Uvinul A Plus by mass, according to the following protocol. In a flask, were introduced 70 g Octyl methoxy cinnamate, 10 g of Uvasorb HER, 10 g of Uvasorb K2A and 10 g of Uvinul A Plus. The mixture thus obtained was heated to 60° C. with stirring until obtaining a clear liquid.

### Formulations

The compositions of Example 1, Example 2. Example 3, Example 4 and Example 5 were compared to known and commercially available anti-color fading compositions, (e.g. COVABSORB (WO2006/005846)), in cosmetic water-based and non-water-based formulations containing dyes and perfumes.

### Formula A—Shampoo

Tables 1 and 2 report the compositions of shampoo formulas:

TABLE 1

| | A1 | A2 | A3 | A4 | A5 | A6 | A7 |
|---|---|---|---|---|---|---|---|
| TEXAPON NSA | 30% | 30% | 30% | 30% | 30% | 30% | 30% |
| COCOAMIDOPROPYLBETAINE | 10% | 10% | 10% | 10% | 10% | 10% | 10% |
| FD & C Red 40 | 0.001% | 0.001% | 0.001% | 0.001% | 0.001% | 0.001% | 0.001% |
| Example 1 | 1% | — | — | — | — | — | — |
| Example 2 | — | 1% | — | — | — | — | — |
| Example 3 | — | — | 1% | — | — | — | — |
| Example 4 | — | — | — | 1% | — | — | — |
| Example 5 | — | — | — | — | 1% | — | — |
| COVABSORB | — | — | — | — | — | 1% | — |
| Water | to 100% | to 100% | to 100% | to 100% | to 100% | to 100% | to 100% |
| pH | 5.1 | 5.2 | 5.5 | 4.9 | 5.1 | 5 | 5.3 |

TABLE 2

| | A8 | A9 | A10 | A11 | A12 | A13 | A14 |
|---|---|---|---|---|---|---|---|
| TEXAPON NSA | 30% | 30% | 30% | 30% | 30% | 30% | 30% |
| COCOAMIDOPROPYLBETAINE | 10% | 10% | 10% | 10% | 10% | 10% | 10% |
| FD & C Red 40 | 0.001% | 0.001% | 0.001% | 0.001% | 0.001% | 0.001% | 0.001% |
| Example 1 | 0.65% | — | — | — | — | — | — |
| Example 2 | — | 0.80% | — | — | — | — | — |
| Example 3 | — | — | 0.80% | — | — | — | — |
| Example 4 | — | — | — | 0.75% | — | — | — |
| Example 5 | — | — | — | — | 0.87% | — | — |
| COVABSORB | — | — | — | — | — | 1.00% | — |
| Water | to 100% | to 100% | to 100% | to 100% | to 100% | to 100% | to 100% |
| pH | 5.2 | 5.2 | 4.9 | 5.1 | 5.2 | 5.3 | 5.3 |

The compounds of the Formulas A7 and A14 did not contain any stabilizing compositions and they were used as control.

### Formula B—Oil Based

Tables 3 and 4 report the compositions of oil based formulas:

TABLE 3

| | B1 | B2 | B3 | B4 | B5 | B6 | B7 |
|---|---|---|---|---|---|---|---|
| D & C Red 17 | 0.001% | 0.001% | 0.001% | 0.001% | 0.001% | 0.001% | 0.001% |
| Example 1 | 1% | — | — | — | — | — | — |
| Example 2 | — | 1% | — | — | — | — | — |
| Example 3 | — | — | 1% | — | — | — | — |
| Example 4 | — | — | — | 1% | — | — | — |
| Example 5 | — | — | — | — | 1% | — | — |
| COVABSORB | — | — | — | — | — | 1% | — |
| Castor Oil | to 100% | to 100% | to 100% | to 100% | to 100% | to 100% | to 100% |

TABLE 4

| | B8 | B9 | B10 | B11 | B12 | B13 | B14 |
|---|---|---|---|---|---|---|---|
| D & C Red 17 | 0.001% | 0.001% | 0.001% | 0.001% | 0.001% | 0.001% | 0.001% |
| Example 1 | 0.65% | — | — | — | — | — | — |
| Example 2 | — | 0.80% | — | — | — | — | — |
| Example 3 | — | — | 0.80% | — | — | — | — |
| Example 4 | — | — | — | 0.75% | — | — | — |
| Example 5 | — | — | — | — | 0.87% | — | — |
| COVABSORB | — | — | — | — | — | 1% | — |
| Castor Oil | to 100% | to 100% | to 100% | to 100% | to 100% | to 100% | to 100% |

The compounds of the Formulas B7 and B14 did not contain any stabilizing compositions and they were used as control.

### Formula C—Perfume

Tables 5 and 6 is reported the compositions of perfume formulas:

TABLE 5

| | C1 | C2 | C3 | C4 | C5 | C6 | C7 |
|---|---|---|---|---|---|---|---|
| Ethylvanillin | 3% | 3% | 3% | 3% | 3% | 3% | 3% |
| FD & C Red 40 | 0.001% | 0.001% | 0.001% | 0.001% | 0.001% | 0.001% | 0.001% |
| Example 1 | 1% | — | — | — | — | — | — |
| Example 2 | — | 1% | — | — | — | — | — |
| Example 3 | — | — | 1% | — | — | — | — |
| Example 4 | — | — | — | 1% | — | — | — |
| Example 5 | — | — | — | — | 1% | — | — |
| COVABSORB | — | — | — | — | — | 1% | — |
| Ethanol 96% | to 100% | to 100% | to 100% | to 100% | to 100% | to 100% | to 100% |

TABLE 6

| | C8 | C9 | C10 | C11 | C12 | C13 | C14 |
|---|---|---|---|---|---|---|---|
| Ethylvanillin | 3% | 3% | 3% | 3% | 3% | 3% | 3% |
| FD & C Red 40 | 0.001% | 0.001% | 0.001% | 0.001% | 0.001% | 0.001% | 0.001% |
| Example 1 | 0.65% | — | — | — | — | — | — |
| Example 2 | — | 0.80% | — | — | — | — | — |
| Example 3 | — | — | 0.80% | — | — | — | — |
| Example 4 | — | — | — | 0.75% | — | — | — |
| Example 5 | — | — | — | — | 0.87% | — | — |
| COVABSORB | — | — | — | — | — | — 1% | — |
| Ethanol 96% | to 100% | to 100% | to 100% | to 100% | to 100% | to 100% | to 100% |

The compounds of the Formulas C7 and C14 did not contain any stabilizing compositions and they were used as controls.

### Formula D—Water Based Formula

Tables 7 and 8 is report the compositions of water based formulas:

TABLE 7

| | D1 | D2 | D3 | D4 | D5 | D6 | D7 |
|---|---|---|---|---|---|---|---|
| FD & C Blue 1 | 0.001% | 0.001% | 0.001% | 0.001% | 0.001% | 0.001% | 0.001% |
| Example 1 | 1% | — | — | — | — | — | — |
| Example 2 | — | 1% | — | — | — | — | — |
| Example 3 | — | — | 1% | — | — | — | — |
| Example 4 | — | — | — | — 1% | — | — | — |
| Example 5 | — | — | — | — | 1% | — | — |
| COVABSORB | — | — | — | — | — | 1%% | — |
| PPG-26-Buteth-26 | 2% | 2% | 2% | 2% | 2% | 2% | 2% |
| PEG-40 Hydrogenated castor oil | 2% | 2% | 2% | 2% | 2% | 2% | 2% |
| Water | to 100% | to 100% | to 100% | to 100% | to 100% | to 100% | to 100% |
| pH | 4.9 | 5.1 | 5.2 | 5.1 | 5.2 | 4.9 | 5.2 |

TABLE 8

| | D8 | D9 | D10 | D11 | D12 | D13 | D14 |
|---|---|---|---|---|---|---|---|
| FD & C Blue 1 | 0.001% | 0.001% | 0.001% | 0.001% | 0.001% | 0.001% | 0.001% |
| Example 1 | 0.65% | — | — | — | — | — | — |
| Example 2 | — | 0.80% | — | — | — | — | — |
| Example 3 | — | — | 0.81% | — | — | — | — |
| Example 4 | — | — | — | 0.75% | — | — | — |
| Example 5 | — | — | — | — | 0.87% | — | — |
| COVABSORB | — | — | — | — | — | 1% | — |
| PPG-26-Buteth-26 | 2% | 2% | 2% | 2% | 2% | 2% | 2% |
| PEG-40 Hydrogenated castor oil | 2% | 2% | 2% | 2% | 2% | 2% | 2% |
| Water | to 100% | to 100% | to 100% | to 100% | to 100% | to 100% | to 100% |
| pH | 5.1 | 4.8 | 5.1 | 5.2 | 5.2 | 5.1 | 5.3 |

The compounds of the Formulas D7 and D14 did not contain any stabilizing compositions as they were used as controls.

### Colorimetric Measures and WOM

The cosmetic formulations described above were exposed to UV rays in a WOM ATLAS Ci4000 apparatus and monitored over time during the exposure. The stabilizing effect of the compositions was demonstrated by measurement of the decay of the absorbance at the max wavelength of absorbance between 400 and 720 nm at the max wavelength characteristic of the relative dye, or by measuring the time of exposure to obtain the same absorbance at the max wavelength characteristic of the relative dye. Both stabilizing effects were compared to the known and commercially available anti-color fading compositions, (e.g. COVABSORBI (WO2006/005846)). The colorimetric strength (CS) of each cosmetic formulation is determined by the following mathematical formula:

CS=(Amax exposed/Amax control)×100

CS is the colorimetric strength expressed in percent, Amax control is the max absorbance of the control not exposed at the max wavelength characteristic of the relative dye and Amax exposed is the absorbance of the formula after the time of exposure. A spectrophotometer UV/Vis Perkin Elmer Lambda 25 was used to determine the absorbance prior, during and after the exposure. CS equal to 100% when Amax exposed=Amax control, evidencing that the dye has been stabilized, CS equal to 0 when the Amax exposed is zero and the dye is completely deteriorated.

The cosmetic formulations of Tables 2, 4, 6 and 8 were exposed to the UV rays into a WOM ATLAS Ci4000 for 24 hours. I was surprisingly found 15-35% less amount of stabilizing composition shown in Example 1, 2, 3, 4 and 5 was required to obtain characteristics substantially equivalent to or better than known commercial available COVABSORB (WO 2006/005846). Such characteristics include protection from UV radiation.

TABLE 9

| | A8 | A9 | A10 | A11 | A12 | A13 | A14 |
|---|---|---|---|---|---|---|---|
| CS | 88% | 86% | 86% | 87% | 85% | 85% | 0% |
| | B8 | B9 | B10B | B11 | B12 | B13 | B14 |
| CS | 87% | 85% | 87% | 84% | 83% | 82% | 0% |
| | C8 | C9 | C10 | C11 | C12 | C13 | C14 |
| CS | 90% | 86% | 87% | 86% | 86% | 86% | 0% |
| | D8 | D9 | D10 | D11 | D12 | D13 | D14 |
| CS | 84% | 83% | 84% | 82% | 81% | 80% | 0% |

According to the result shown in Table 9, the compounds of the formulas A8, B8, C8 and D8 comprises 35% less amount of stabilizing composition than compounds of the formulas A13, B13, C13 and D13 (COVABSORB) to obtain at least substantially equivalent levels of UV radiation protection.

The compounds of the formulas A9, B9, C9 and D9 comprised 20% less amount of stabilizing composition than A13, B13, C13 and D13 (COVABSORB) to obtain at least substantially equivalent levels of UV radiation protection.

The compounds of the formulas A10, B10, C10 and D10 comprised 20% less amount of stabilizing composition than A13, B13, C13 and D13 (COVABSORB) to obtain at least substantially equivalent levels of UV protection.

The compounds of the formulas A11, B11, C11 and D11 comprised 25% less amount of stabilizing composition than A13, B13, C13 and D13 (COVABSORB) to obtain at least substantially equivalent levels of UV protection.

The compounds of the formulas A12, B12, C12 and D12 comprised 13% less amount of stabilizing composition than A13, B13, C13 and B13 (COVABSORB) to obtain at least substantially equivalent levels of UV protection.

The cosmetic formulation of the Table 1, 3, 5 and 7 were exposed to the UV rays in a WOM ATLAS Ci4000. The CS was determined for all the cosmetic formulations every 4 hours. It was surprisingly found that the stabilizing composition of Examples 1, 2, 3, 4 and 5 remained effective for UV radiation protection for an increased duration of from about 15 to 50% as compared to known, commercially available COVABSORB (WO 2006/005846) to obtain the same CS.

TABLE 10

| | A1 | A2 | A3 | A4 | A5 | A6 | A7 |
|---|---|---|---|---|---|---|---|
| Hours | 36 | 30 | 31 | 31 | 27 | 24 | 0% CS₂₄ₜₕ |
| | B1 | B2 | B3 | B4 | B5 | B6 | B7 |
| Hours | 35 | 31 | 30 | 31 | 28 | 24 | 0% CS₂₄ₜₕ |
| | C1 | C2 | C3 | C4 | C5 | C6 | C7 |
| Hours | 37 | 30 | 30 | 32 | 28 | 24 | 0% CS₂₄ₜₕ |
| | D1 | D2 | D3 | D4 | D5 | D6 | D7 |
| Hours | 36 | 29 | 31 | 32 | 27 | 24 | 0% CS₂₄ₜₕ |

According to Table 10, the formulas A1, B1, C1 and D1 remained effective for UV radiation protection for an increased duration of from about 15 to 50% as compared to known, commercially available COVABSORB (WO 2006/005846) to obtain a substantially equivalent level of UV radiation protection.

The compounds of the formulas A2, B2, C2 and D2 remained effective for UV radiation protection for an increased duration of from about 25% as compared to known, commercially available COVABSORB (WO 2006/005846) to obtain a substantially equivalent CS.

The compounds of the formulas A3, B3, C3 and D3 remained effective for UV radiation protection for an increased duration of from about 25% as compared to known, commercially available COVABSORB (WO 2006/005846) to obtain a substantially equivalent level of protection.

The compounds of the formulas A4, B4, C4 and D4 remained effective for UV radiation protection for an increased duration of from about 30% as compared to known, commercially available COVABSORB (WO 2006/005846) to obtain a substantially equivalent level of protection.

The compounds of formulas A5, B5, C5 and D5 remained effective for UV radiation protection for an increased duration of from about 15% as compared to known, commercially available COVABSORB (WO 2006/005846) to obtain a substantially equivalent level of protection.

### HPLC Measures and WOM

The cosmetic formulations C (C1 to C14) described above were exposed to UV rays in a WOM ATLAS Ci4000 apparatus and monitored over time during the exposure. The stabilizing effect of the composition was demonstrated by HPLC, quantifying by mass the amount of the Ethylvanillin in the formulation prior, during and after the exposure to UV rays. The UV stabilizing effects were compared to the known, commercially available anti-color fading compositions, (e.g. COVABSORB (WO2006/005846)). An HPLC Agilent Technologies series 1200 with UV-Vis detector was used to quantify the amount of Ethylvanillin present in the formulations.

The cosmetic formulations of Table 6 were exposed to the UV rays in a WOM ATLAS Ci4000 for 24 hours and it was surprisingly found that a reduced amount of from about 15 to about 35% of stabilizing compositions of Example 1, 2, 3, 4 and 5 was required to achieve a substantially equivalent level of UV radiation protection (duration) as compared to the known, commercially available COVABSORB (WO 2006/005846).

TABLE 11

| | C8 | C9 | C10 | C11 | C12 | C13 | C14 |
|---|---|---|---|---|---|---|---|
| Ethyl- | 2.72% | 2.69% | 2.73% | 2.71% | 2.65% | 2.70% | 0% |
| vanil- | | | | | | | |
| lin % w/w | | | | | | | |

The compounds of formula C8 used 35% less amount of stabilizing composition than C13 (COVABSORB) to obtain a substantially equivalent level of UV protection.

The compounds of formula C9 used 20% less amount of stabilizing composition than C13 (COVABSORB) to obtain a substantially equivalent level of UV protection.

The compounds of formula C10 used 20% less amount of stabilizing composition than C13 (COVABSORB) to obtain a substantially equivalent level of UV protection.

The compounds of formula C11 used 25% less amount of stabilizing composition than C13 (COVABSORB) to obtain a substantially equivalent level of UV protection.

The compounds of formula C12 used 13% less amount of stabilizing composition than C13 (COVABSORB) to obtain a substantially equivalent level of UV protection.

The cosmetic formulations presented in Table 5 were exposed to the UV rays in a WOM ATLAS Ci4000. The Ethylvanillin content was determined by HPLC for the cosmetic formulations every 4 hours. It was surprisingly found that stabilizing compositions presented in Example 1, 2, 3, 4 and 5 remain effective for UV radiation protection for an increased duration of from about 15 to about 50% as compared to known, commercially available COVABSORB (WO 2006/005846) to obtain a substantially equivalent CS.

TABLE 12

| | C1 | C2 | C3 | C4 | C5 | C6 | C7 |
|---|---|---|---|---|---|---|---|
| Hours | 36 | 29 | 31 | 31 | 28 | 24 | 0% Ethylvan, HPLC ₂₄ₜₕ |

The hours reported in Table 12 represent the WOM hours needed to achieve a 2.7-2.8% w/w content of Ethylvanillin in the formulations.

The compounds of formula C1 remained effective for UV radiation protection for an increased duration of from about 50% as compared to known, commercially available COVABSORB (WO 2006/005846) C6 to obtain a substantially equivalent level of protection.

The compounds of formula C2 remained effective for UV radiation protection for an increased duration of from about 25% as compared to known, commercially available COVABSORB (WO 2006/005846) C6 to obtain a substantially equivalent level of protection.

The compounds of formula C3 remained effective for UV radiation protection for an increased duration of from about 25% as compared to known, commercially available COVABSORB (WO 2006/005846) C6 to obtain a substantially equivalent level of protection.

The compounds of formula C4 remained effective for UV radiation protection for an increased duration of from about 30% as compared to known, commercially available COVABSORB (WO 2006/005846) C6 to obtain a substantially equivalent level of protection.

The compounds of formula C5 remained effective for UV radiation protection for an increased duration of from about 15% as compared to known, commercially available COVABSORB (WO 2006/005846) C6 to obtain a substantially equivalent level of protection.

When introducing elements of the present disclosure or exemplary aspects or embodiment(s) thereof, the articles “a,” “an,” “the” and “said” are intended to mean that there are one or more of the elements. The terms “comprising.” “including” and “having” are intended to be inclusive and mean that there may be additional elements other than the listed elements. Although this disclosure has been described with respect to specific embodiments, the details of these embodiments are not to be construed as limitations. While the preferred variations and alternatives of the present disclosure have been illustrated and described, it will be appreciated that various changes and substitutions can be made therein without departing from the spirit and scope of the disclosure.

What is claimed is:

1. A method for stabilizing the color and fragrance of a cosmetic against the effect of UV radiation comprising the steps of:
preparing a UV absorbing composition comprising:
(a) at least one cinnamate-containing compound or cinnamate-containing derivative compound, said cinnamate-containing compound or cinnamate-containing derivative compound absorbing radiation in the UV region;
(b) at least one triazine-containing compound or triazine-containing derivative compound, said triazine-containing compound or triazine-containing derivative compound absorbing radiation in the UVB region; and
(c) at least one dibenzoylmethane-containing compound or dibenzoylmethane-containing derivative compound, said dibenzoylmethane-containing compound or dibenzoylmethane-containing derivative compound absorbing radiation in the UV region; and
incorporating the UV absorbing composition into a cosmetic formulation.

2. The method of claim 1, wherein the UV absorbing composition comprises about 5.0 to about 90.0% by weight of compound (a); about 5.0 to about 90.0% by weight of compound (b); and about 5.0 to about 90.0% by weight of compound (c).

3. The method of claim 1, wherein compound (a) comprises Uvasorb HEB, CAS [154702-15-5]; compound (b) comprises Octyl methoxycinnamate, CAS [5466-77-3]; and compound (c) comprises Avobenzone, CAS [70356-09-1].

4. The method of claim 1, wherein the UV absorbing composition further comprises (d) one or more surfactants.

5. The method of claim 4, wherein the UV absorbing composition comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

6. The method of claim 4, wherein the composition comprises a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Avobenzone, CAS [70356-09-1]; and about 20.0 to about 80.0% by weight of compound (d).

7. A method for stabilizing the color and fragrance of a cosmetic against the effect of UV radiation comprising the steps of:
preparing a UV absorbing composition comprising:
(a) at least one UV absorbing compound, said UV absorbing compound comprising a cinnamate-containing compound or a cinnamate-containing derivative compound;
(b) at least one UV absorbing compound absorbing in the UVB region, said UV absorbing compound absorbing in the UVB region comprising a triazines-containing compound or a triazine-containing derivative compound; and
(c) at least one UV absorber compound absorbing in the UVA region, said UV absorber compound absorbing in the UVA region comprising a triazine-containing compound or a triazine derivative compound; and
incorporating the UV absorbing composition into a cosmetic formulation.

8. The method of claim 7, wherein the UV absorbing composition comprises a combination of: about 5.0 to about 90.0% by weight of compound (a); about 5.0 to about 90.0% by weight of compound (b); and about 5.0 to about 90.0% by weight of compound (c).

9. The method of claim 7, wherein compound (a) comprises Uvasorb HEB, CAS [154702-15-5]; compound (b) comprises Octyl methoxycinnamate, CAS [5466-77-3]; and compound (c) comprises Uvasorb K2A, CAS [288254-16-0].

10. The method of claim 7, wherein the UV absorbing composition further comprises: (d) at least one surfactant.

11. The method of claim 7, wherein the UV absorbing compound comprises a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

12. The composition of claim 10, wherein the composition comprises a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or a mixture of surfactants.

13. A composition, comprising:
(a) at least one UV absorbing compound comprising a cinnamate-containing compound or a cinnamate-containing derivative compound;
(b) at least one IV absorbing compound absorbing in the UVB region, said UV absorbing compound absorbing in the UVB region comprising a triazine-containing compound or a triazine-containing derivative compound; and
(c) at least one UV absorbing compound comprising a dibenzoylmethane-containing compound or a dibenzoylmethane-containing derivative compound.

14. The composition of claim 13, wherein the composition comprises a combination of: about 5.0 to about 90.0% by weight of compound (a); about 5.0 to about 90.0% by weight of compound (b); and about 5.0 to about 90.0% by weight of compound (c).

15. The composition of claim 13, comprising a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

16. The composition of claim 13, wherein compound (a) comprises Uvasorb HEB, CAS [154702-15-5]; compound (b) comprises Octyl methoxycinnamate, CAS [5466-77-3]; and compound (c) comprises Avobenzone, CAS [70356-09-1].

17. The composition of claim 13, wherein the composition further comprises:
(d) one or more surfactants.

18. A composition, comprising:
(a) at least one UV absorbing compound comprising a cinnamate-containing compound or cinnamate-containing derivative compound;
(b) at least one UV absorbing compound absorbing radiation in the UVB region, said UV absorbing compound absorbing radiation in the UVB region comprising a triazine-containing compound or a triazine-containing derivative compound; and
(c) at least one UV absorbing compound absorbing radiation in the UVA region, said UV absorbing compound absorbing radiation in the UVA region comprising a triazine-containing compound or a triazine-containing derivative compound.

19. The composition of claim 18, further comprising (d) one or more surfactants.

20. The composition of claim 19, comprising a combination of: about 1.0 to about 50.0% by weight of compound (a); about 1.0 to about 50.0% by weight of compound (b); about 1.0 to about 50.0% by weight of compound (c); and about 50.0 to about 90.0% by weight of compound (d).

21. The composition of claim 18, comprising a combination of: about 1.0 to about 20.0% by weight of Uvasorb HEB, CAS [154702-15-5]; about 1.0 to about 20.0% by weight of Octyl methoxycinnamate, CAS [5466-77-3]; about 1.0 to about 20.0% by weight of Uvasorb K2A, CAS [288254-16-0]; and about 20.0 to about 80.0% of one or more surfactants.

22. The composition of claim 18, comprising a combination of: about 5.0 to about 90.0% by weight of compound (a); about 5.0 to about 90.0% by weight of compound (b); and about 5.0 to about 90.0% by weight of compound (c).

23. The composition of claim 18, wherein: compound (a) comprises Uvasorb HEB, CAS [154702-15-5]; compound (b) comprises Octyl methoxycinnamate, CAS [5466-77-3]; and compound (c) compound Uvasorb K2A, CAS [288254-16-0].

24. A cosmetic formulation or cosmetic precursor formulation comprising the composition of claim 18.

25. A household product formulation or household product precursor formulation comprising the composition of claim 18.
